(19) United States
(12) Patent Application Publication
(10) Pub. No.: US 2009/0024164 A1
(43) Pub. Date: Jan. 22, 2009

(54) System for determining spinal implants

(76) Inventor: Seth L. Neubardt, Mamaroneck, NY (US)

Correspondence Address: LAW OFFICE OF LEO ZUCKER, 2591 Duning Drive, PO BOX 1177, Yorktown Heights, NY, 10598, US

(21) Appl. No.: 12/215,097

(22) Filed: Jun. 25, 2008

Related U.S. Application Data
(60) Provisional application No. 60/937,055, filed on Jun. 25, 2007.

(51) Int. Cl. A61F 5/00

(52) U.S. Cl. 606/242; 606/237

(57) ABSTRACT

A system for determining spinal implants includes a surgical table that moves a patient secured on the table into different positions in response to patient command, including a position of pain (POP) where the patient experiences the most pain from one or more affected levels of the spine, and a position of comfort (POC) where the patient has the least amount of pain. A scanner scans the patient on the table at least at the POP and the POC, and produces corresponding radiographic image data. Image processing apparatus coupled to the scanner quantifies changes in the relative position of adjacent vertebra at each affected level when the vertebra move from their configuration in the patient's POP, to their configuration in the patient's POC. The apparatus can then select an optimal implant device from among a number of devices whose characteristics are stored in a system database.

### CROSS REFERENCE TO RELATED APPLICATION

This application claims priority under 35 U.S.C. § 119(e) of U.S. Provisional Patent Application No. 60/937,055 filed Jun. 25, 2007, and entitled “System for Treatment of Spinal Abnormalities Using Patient Selected Positions”.

### BACKGROUND OF THE INVENTION

1. Field of the Invention

The present invention concerns systems for treating spinal abnormalities by the use of implant devices.

2. Discussion of the Known Art

Surgeons and clinical practitioners use radiographic tools such as MRI scans and X-rays, together with their experience and intuition to evaluate if a patient's spine is configured within a so-called “normal” range. If not, the surgeon then decides how much restoration or positional variance must be imparted to the spine to eliminate or reduce back pain using procedures such as, e.g., spinal fusion or disc replacement. Because any given surgeon's intuition is usually based on his or her training and the fellowship program they completed, the current practice of evaluating patients' spines and identifying implant devices to treat suspected abnormalities is fraught with uncertainties, and it results in patient outcomes that vary widely.

Further, practitioners today are concerned not just with a “normal” range of spinal configurations, but also with those static positions that cause the patient to experience pain. For example, a patient may have a normal range of motion but still feel pain. And pain alone can not be detected by way of an x-ray or scan.

U.S. Pat. No. 6,708,693 (Mar. 23, 2004) discloses a method and a device for positioning a patient during MRI imaging diagnosis. The patient lies supine on a platform with their legs extended and feet in contact with a footrest, and a harness is worn above the area of the spine to be compressed and imaged. A pair of straps fixed to the harness pull the latter toward the footrest, thereby compressing and flexing the patient's spine as desired for imaging. U.S. Pat. No. 6,860,272 (Mar. 1, 2005) and U.S. Pat. Appl'n Pub. No. 2005/0165293 (Jul. 28, 2005) relate to a device having an adjustable footplate for immobilizing a patient and compressing the patient's skeleton, joints and/or spine during imaging.

U.S. Pat. Appl'n Pub. No. 2005/0177239 (Aug. 11, 2005) discloses a method and apparatus for computerized spinal surgery with an implant device having an inflatable cavity for placement between end plates of adjacent vertebra. The publication also discloses (at pars. 147-48 and 995-98, & FIGS. 42 & 43) a surgical procedure wherein the patient is mounted on a support table, a region of the patient's spine is imaged, a 3-D image file of the region is obtained and stored, and the file is used for planning and carrying out computer-controlled implant surgery.

As far as is known, however, no procedure or system has been proposed wherein a level of a patient's spine is scanned while the patient assumes a position where he or she feels a greatest amount of discomfort, and the same level is scanned again while the patient assumes a position that yields a greatest amount of comfort, and a device is identified to be implanted at the scanned level to maintain the patient's comfort in accordance with the scan results.

### SUMMARY OF THE INVENTION

According to invention, a system for determining spinal implants includes a surgical table arranged to move a patient when secured on the table into a number of different positions in response to commands by the patient, the positions including a position of pain (POP) where the patient experiences maximum pain from one or more affected levels of his or her spine, and a position of comfort (POC) where the patient experiences a least amount of pain from the affected levels. A scanner operatively associated with the table is arranged to scan the patient at least at the POP and the POC, and to produce corresponding radiographic image data corresponding to the patient's spine. Image processing apparatus is coupled to the scanner and is configured to quantify changes in relative position of adjacent vertebra at each affected level when the vertebra move from a configuration corresponding to the patient's POP to a configuration corresponding to the patient's POC.

In the disclosed embodiment, an implant device database is associated with the image processing apparatus, and the database is configured to store information concerning characteristics of a number of implant devices selected from the group comprising artificial discs, rod/screw constructs, flexible rods, and distraction devices, and the image processing apparatus is configured to select one or more implant devices from among those whose characteristics are stored in the database according to the quantified changes in position of the vertebra at each affected level.

For a better understanding of the invention, reference is made to the following description taken in conjunction with the accompanying drawing and the appended claims.

### BRIEF DESCRIPTION OF THE DRAWING

In the drawing:

FIGS. 1 to 6 chart various steps to be taken when using a system for determining spinal implants according to the invention;

FIG. 7 is a schematic block diagram of a system for determining spinal implants according to the invention; and

FIGS. 8 to 20 illustrate two hypothetical case studies in which the inventive system may be applied.

### DETAILED DESCRIPTION OF THE INVENTION

The present invention is a system for identifying an optimal spinal implant for a given patient. Once a physician identifies a level of the patient's spine that is acting as a source of discomfort, an appropriate implant is determined based on images of the relative configuration of the vertebra at the affected level taken at (i) a position of discomfort or pain (POP) whereat the patient reports experiencing the greatest amount of back pain, and (ii) a position of comfort (POC) at which the patient feels most comfortable. The relative positions of adjacent vertebra at the affected level for the POP and the POC may be scanned using known tools, for example, fluoroscopy, x-ray, or CT scans, that are capable of yielding quantitative measurements of spinal distraction, compression, flexion, extension, lateral bending, and rotation.

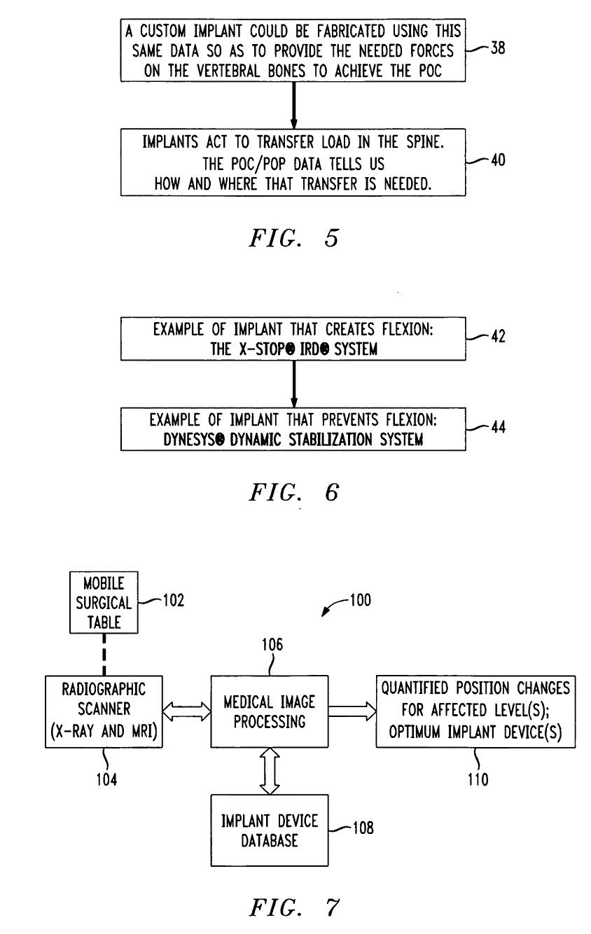

One or more devices are then identified which when implanted at the affected level of the patient's spine, will urge the vertebra at the level into the configuration measured at the patient's POC, and inhibit the vertebra from movement to the configuration measured at the patient's POP. For example, if the patient's POC is such that the configuration of the vertebra at the affected level is one of flexion (rather than “normal”), then a device that creates flexion is implanted at that level. An example of such an device is the X-Stop® IPD® System. See step 42 in FIG. 5. And if the patient's POP is such that the configuration of the vertebra at the affected level is one of flexion, then a device that will prevent flexion (e.g., the Dynesys® Dynamic Stabilization System) is implanted. See step 44 in FIG. 6. Accordingly, the inventive system acknowledges that patients may experience back pain even though clinical examinations and scans of their spinal vertebra may be normal, and the system identifies a patient's POP as a position to avoid within any spectrum of spinal motion that can otherwise be tolerated at the affected level.

Patients with back pain frequently move their bodies into a position of comfort by contorting, bending, or distracting their torso in order to obtain their POC. And their position of maximal pain (the POP) often differs greatly from their POC. When studied radiographically (with x-rays or MRI), the two different positions may be compared, for example, by looking at the anatomic position of each vertebra in relation to the adjacent vertebra. In the inventive system, the difference in anatomic position of the vertebral bones is documented by one or more known techniques such as x-ray or MRI, and then measured. A slight change in the relative position of the adjacent vertebra at the affected level is recorded (e.g., digitally) and analyzed to create a motion model that illustrates the positions through which the spinal bones move from the POC to the POP.

Surgery is then planned to transfer the spinal bones into the relative configuration revealed by the scan for the patient's POC, and to maintain the bones in that configuration. This is accomplished, for example, either by fixing the bones in the desired configuration (e.g., a spinal fusion), or by using a device that allows motion but with such constraint as to avoid the configuration scanned for the patient's POP (e.g., by implanting an artificial disc replacement). For example, a patient that demonstrates maximal pain in flexion (bending forward) should receive a spinal implant that urges the vertebra at the affected level toward spinal extension (bending backward). Given the current availability of spinal implants with various physical characteristics, it is likely that the surgeon will be able to select an appropriate implant to achieve the desired results.

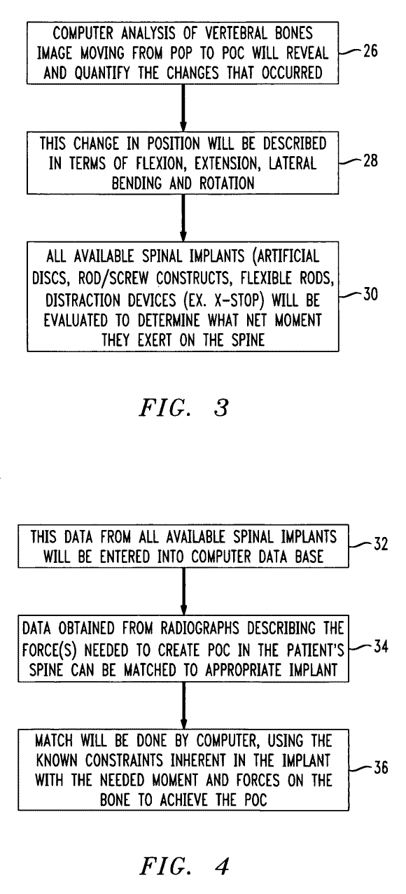

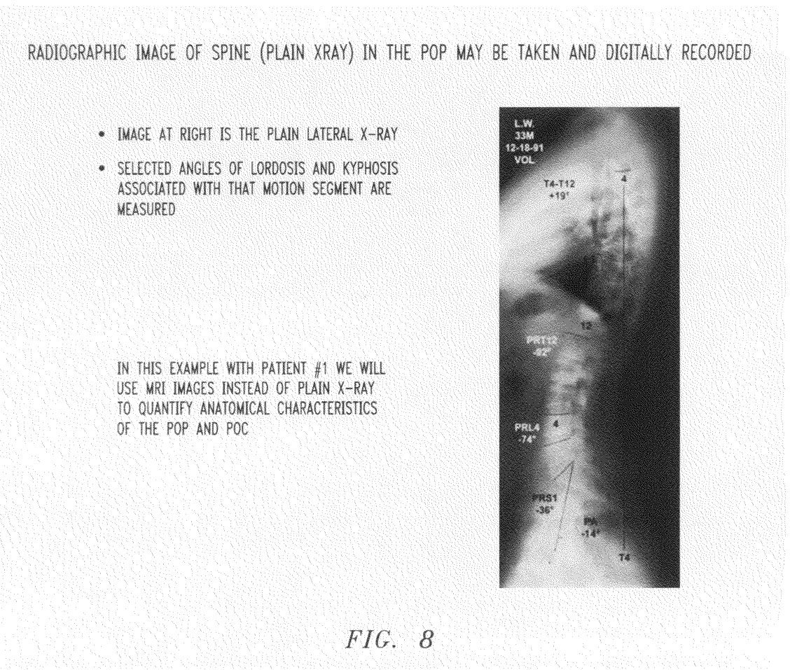

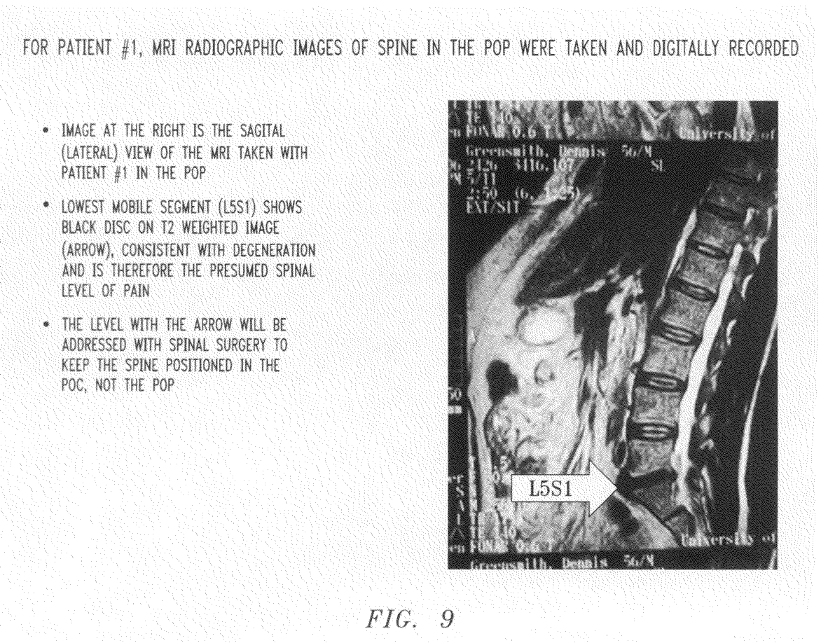

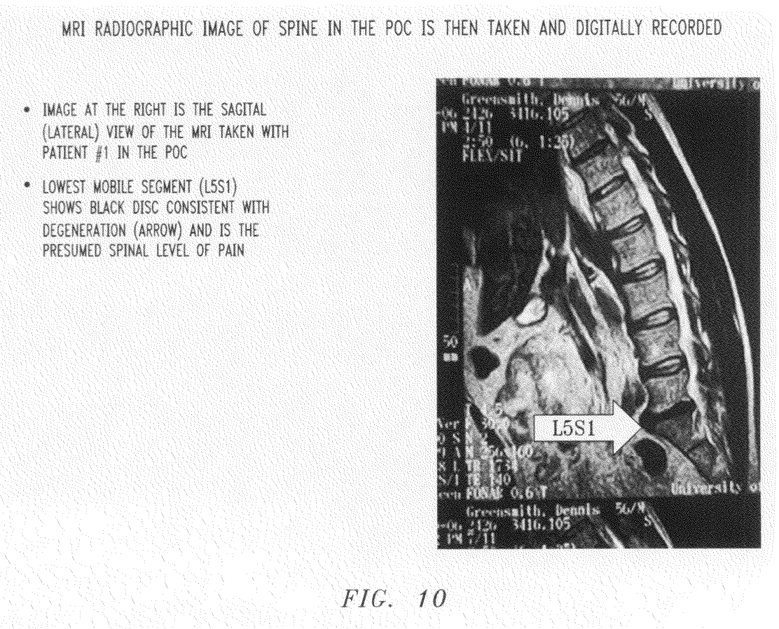

As mentioned, different spinal implants allow for certain kinds of motion while constraining others. According to one aspect of the invention, data representing position and motion profiles for various spinal implants are entered into a database. See steps 30 and 32 in FIGS. 3 and 4, discussed below. A system processing unit (see FIG. 7 and related text, below) having access to the database then selects an optimal implant to achieve a desired alignment of the vertebra at the affected level of a given patient's spine, according to image data representing scans taken at the level for the patient's POP and POC.

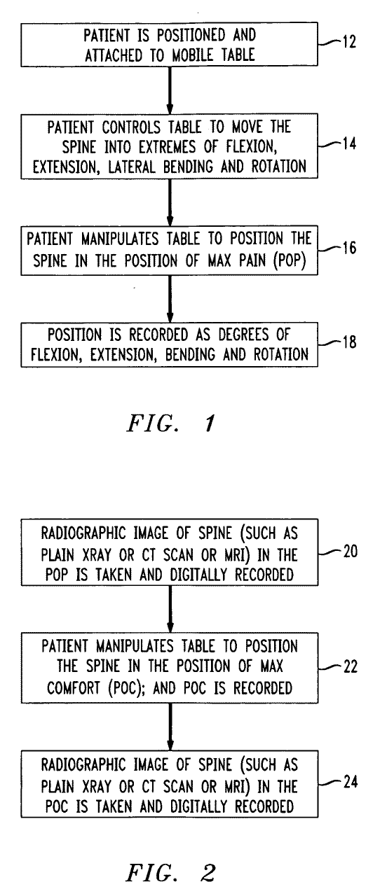

In one scenario, a patient complaining of back pain is initially evaluated to determine if surgery is medically indicated. If so, the surgeon identifies the anatomic level(s) of the patient's spine that are the source of the pain using, e.g., a known discogram procedure that irritates each suspect level and monitors patient response. As illustrated by the chart of FIGS. 1 to 6, the patient is then secured on a table (step 12) constructed and arranged to move him or her into a number of different positions under the control of the patient (step 14). For example, a so-called SpineSix® table system available from MediCepts of Stuart, Fla. 34994. See, U.S. Pat. No. 6,692,451 (Feb. 17, 2004). The table should also be radiolucent or otherwise transparent to radiation that is present when scanning is performed.

The patient reports their position of maximal discomfort (POP) in step 16 and their position of maximal comfort (POC) in step 22. The two table positions are recorded (steps 18 and 22) and corresponding position data is saved in a system memory. Scans, e.g., CT scans, are taken of the affected level(s) for at least the patient's POP (step 20) and POC (step 24). The two scans may be visually compared with one another by the surgeon, and corresponding image data is entered into the system in steps 20 and 24.

In steps 26 and 28, the change in the configuration of the patient's spine at the affected level in order for the patient to be without pain is then determined in terms of such parameters as spinal distraction (elongation), compression (loading), flexion (bending forward), extension (bending backward), lateral bending, translation and rotation. Data corresponding to the change in the spine configuration may be produced by medical image processing apparatus such as disclosed in, e.g., U.S. Pat. No. 7,231,073 (Jun. 12, 2007) all relevant portions of which are incorporated by reference. Based on this data, one or more of spinal implants in the system database are matched with the affected level(s) of the patient's spine. See steps 34, 36 and 40 in FIGS. 4-5. As noted in step 38 in FIG. 5, the same data can be used to fabricate a custom implant that would provide the forces needed to urge the vertebra at the affected level toward the desired configuration at the patient's POC. Once a match is found, a determination is made as to whether or not the implant would tend to allow the vertebra at the affected level to assume the configuration scanned for the patient's POP. If so, the system searches for other potential implants until one having the required motion constraint is identified for the surgeon.

A significant advantage of the invention is improved patient outcome that results from allowing the patient to report directly concerning his or her own POP and POC, while the surgeon is assured of the patient's own interpretation through objective measures such as radiography. That is, an important feature of the inventive system is allowing the pain threshold to be set by the patient and effectively recording the threshold using, e.g., radiographic means and/or a table position. This information is then used intraoperatively as a gauge to compare and judge the ideal pain free state of the patient's spine.

A conventional surgical table may also be used to perform the POP/POC diagnostic test. The patient may position himself or herself preoperatively on the table immediately before surgery, after motion control apparatus associated with the table “remembers” the patient's POC. Once the patient is under anesthesia, the apparatus would then move the table into the position of comfort, thus guaranteeing that the spine will be fused or fixed in the position of comfort.

An intraoperative imaging scan may be taken to check the spinal position, and this information entered into the table control apparatus to move the table in such a way as to recreate the POC in the spine. Intraoperative tools for navigation (such as, e.g., the Medtronic Stealth System) use input information derived from preoperative imaging, intraoperative imaging, and reference points acquired by the surgeon from the surgical field. The position of comfort may also be entered into the navigation system preoperatively, and appear as an overlay on a computer monitor screen so that the surgeon can clearly see his/her goal to change the position of the spinal bones into the position of comfort. Once the POC has been achieved, the bones may be fixed or fused in that position. Likewise, if a motion implant such as an artificial disc is being implanted, then the surgeon can be sure the implant is holding the spine in the desired position of comfort.

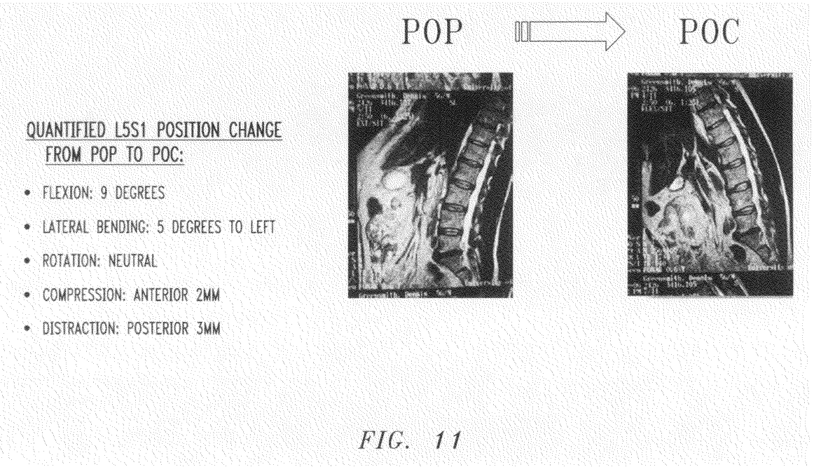

FIG. 7 is a schematic block diagram of one embodiment of a system 100 for determining spinal implants according to the invention, and FIGS. 8 to 20 illustrate two hypothetical case studies or examples wherein the system 100 may be applied to identify an implant that will obtain the best clinical result for each patient.

### EXAMPLE ONE

Patient 1 is positioned and attached to a mobile surgical table 102 such as, e.g., the SpineSix table mentioned above. The patient controls the table 102 to move his/her spine into extremes of flexion, extension, bending, rotation, distraction and compression. The patient then manipulates the table to position their spine in a position where he or she experiences maximum pain (POP). The table position is recorded in terms of degrees of flexion, extension, bending, rotation, distraction and compression.

FIG. 8 is a radiographic image (plain x-ray) of the patient's spine in the POP, obtained from a scanner 104 in FIG. 7. The image, which may be taken and recorded digitally, is a plain lateral x-ray in which selected angles of lordosis and kyphosis associated with the motion segment are measured. In this example, MRI images are preferred instead of plain x-rays so that anatomical characteristics of the patient's POP and POC can be quantified once the positions are determined.

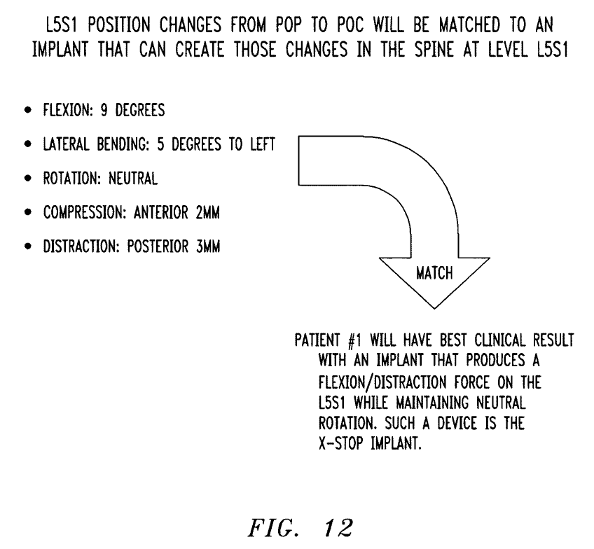

Radiographic MRI images taken by scanner 104 of the patient's spine in the POP are digitally recorded, and FIG. 9 is the sagittal (lateral) view of the MRI taken with the patient in the POP. The lowest mobile segment (L5 S1) shows black disk on T2 weighted image (arrow), consistent with degeneration and is therefore the presumed spinal level of pain. Accordingly, the level with the arrow will be addressed with spinal surgery to keep the spine positioned in the POC as determined below, and not the POP.

Patient 1 then manipulates the table 102 to place their spine in the position of maximum comfort (POC). The table position is again recorded as degrees of flexion, extension, bending, rotation, distraction and compression. This position may be recalled during surgery. An MRI radiographic image of the spine in the POC is taken by scanner 104 and recorded digitally, and FIG. 10 is the sagittal (lateral) view of the MRI taken with the patient in the POC. The lowest mobile segment (L5 S1) shows black disc consistent with degeneration (arrow) and is it the presumed spinal level of pain.

The recorded digital image data is stored and processed using apparatus 106 such as disclosed in the mentioned '073 US patent. Image and data may be recorded and processed using established protocols. For example, measurements may be made using so-called OSIRIS software from the digital imaging unit at the University Hospital of Geneva, Switzerland. Further, a so-called DICOM (Digital Imaging and Communications in Medicine) protocol is a known standard for handling, storing, printing and transmitting information in medical imaging. DICOM includes a file format definition and a network communications protocol.

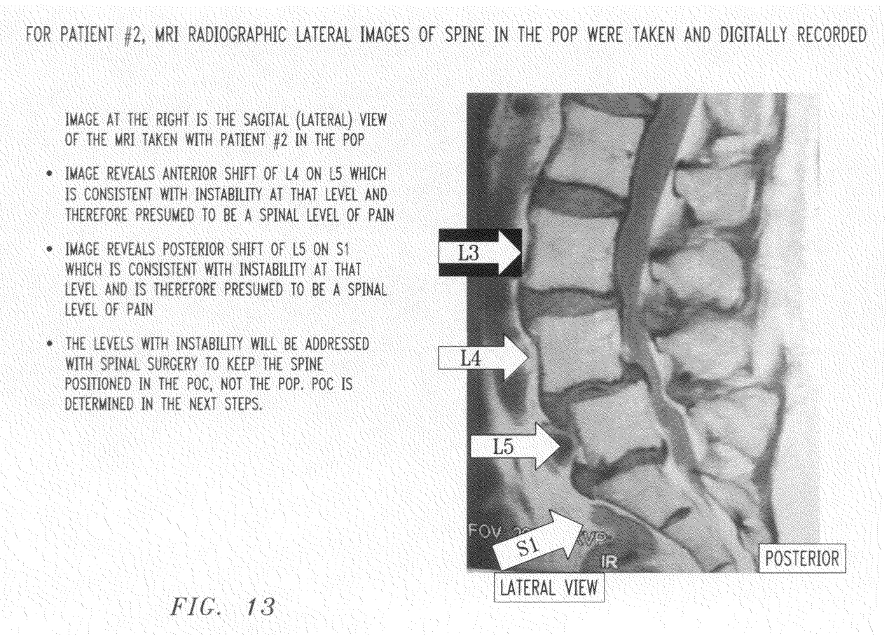

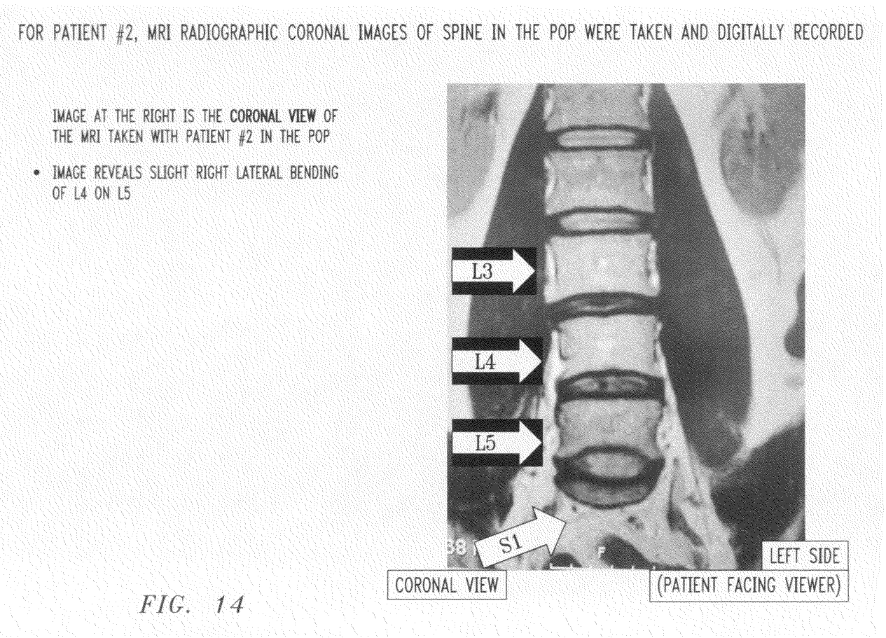

The recorded images of the vertebral bones at the patient's POP and POC reveal and quantify the change in the configuration of the bones in terms of flexion, extension, lateral bending, rotation, translation, compression and distraction. FIG. 11 illustrates the quantified changes in position (in degrees and millimeters) of L5 S1 from the POP to the POC. As shown in FIG. 12, the position changes are matched by the processing apparatus 106 to an implant that can create the changes in the spine at level L5 S1, wherein the implant is selected from among a number of implant devices (e.g., artificial discs, rod/screw constructs, flexible rods and distraction devices) whose characteristics are maintained in a system database 108. In this example, an output 110 of the processing apparatus 106 provides the quantified changes in position, and an indication that patient 1 should have the best clinical results with the above mentioned X-Stop implant which produces a flexion/distraction force on L5 S1 while maintaining neutral rotation.

### EXAMPLE TWO

Patient 2 is positioned and attached to the mobile table 102. The patient controls the table 102 to move his/her spine into extremes of flexion, extension, bending, rotation, distraction and compression. The patient then manipulates the table to position their spine in a position where he or she experiences maximum pain (POP). The table position is recorded in terms of degrees of flexion, extension, bending, rotation, distraction and compression.

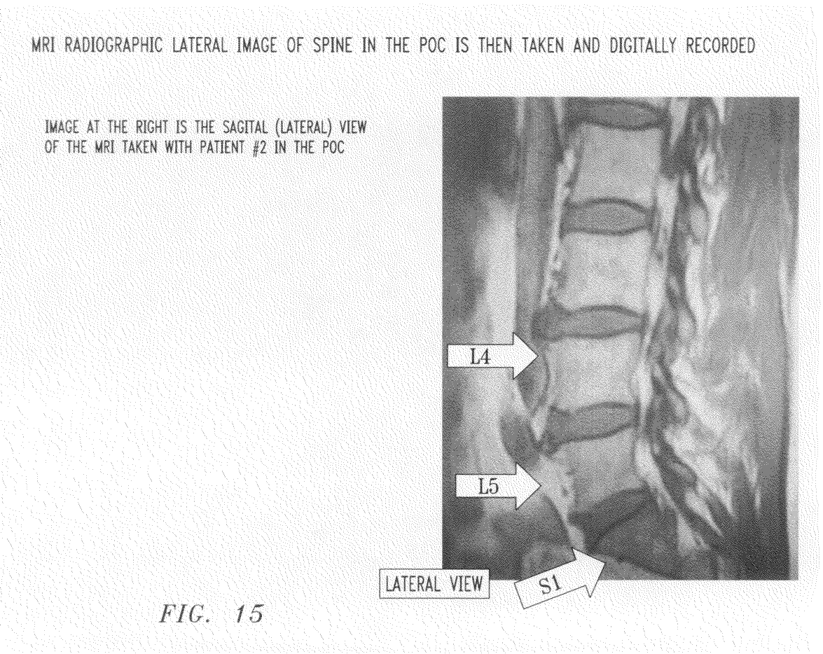

MRI radiographic lateral images of the patient's spine in the POP are taken by the scanner 104 and digitally recorded. FIG. 13 shows the sagittal (lateral) view of the MRI taken with patient 2 in the POP. The image of reveals anterior shift of L4 on L5 which is consistent with instability at that level and therefore presumed to be a spinal level of pain. Further, the image shows a posterior shift of L5 on S1 which is consistent with instability at that level and is therefore also presumed to be a spinal level of pain. Accordingly, the two levels with instability will be addressed with spinal surgery to keep the spine positioned in a POC, not the POP. The POC is determined as follows.

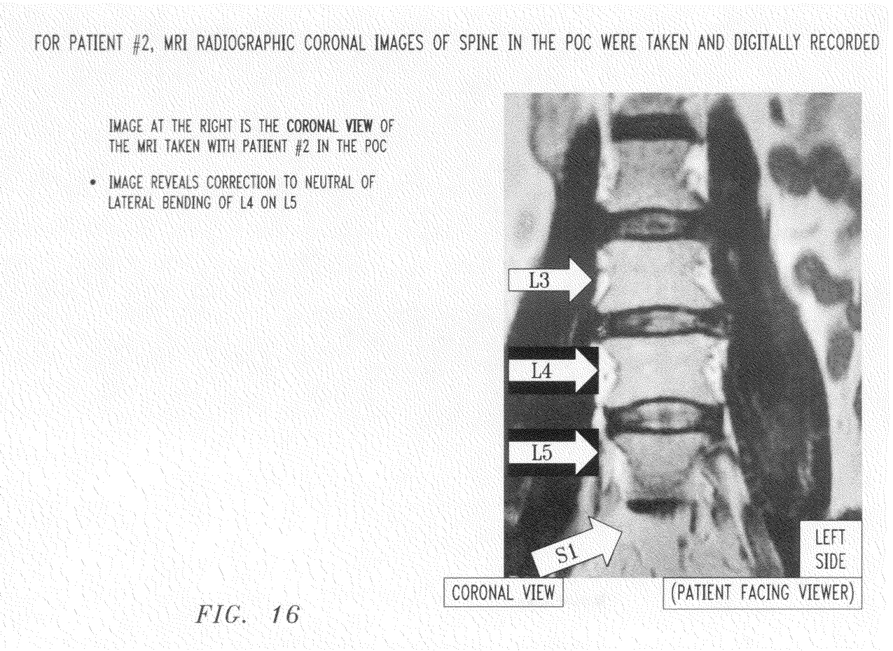

MRI radiographic coronal images of the patient's spine in the POP were also taken by scanner 104 and digitally recorded. FIG. 14 is the coronal view of the MRI with patient 2 in the POP. The image reveals slight right lateral bending of L4 on L5. The patient then manipulates the table 102 to position their spine in the position of maximum comfort or POC. The table position is recorded as degrees of flexion, extension, bending, rotation, distraction and compression. This position may be recalled during surgery.

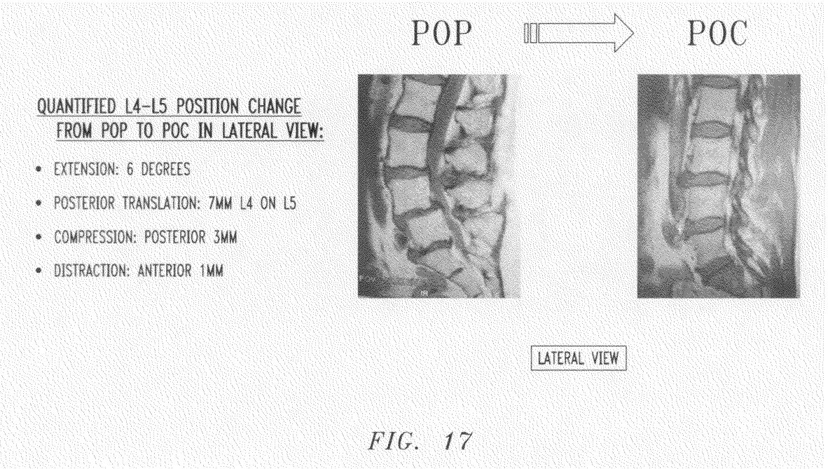

An MRI radiographic lateral image of the spine in the POC is then taken by scanner 104 and digitally recorded. The image in FIG. 15 is the sagittal (lateral) view of the MRI taken with the patient in the POC. MRI radiographic coronal images of the spine in the POC are also taken by scanner 104 and digitally recorded. FIG. 16 is the coronal view of the MRI taken with the patient in the POC. The image reveals correction to neutral of lateral bending of L4 on L5.

The recorded digital image data is stored and processed by the image processing apparatus 106 as in Example ONE. The recorded images of the vertebral bones at the patient's POP and POC reveal and quantify the change in the configuration of the bones in terms of flexion, extension, lateral bending, rotation, translation, compression and distraction.

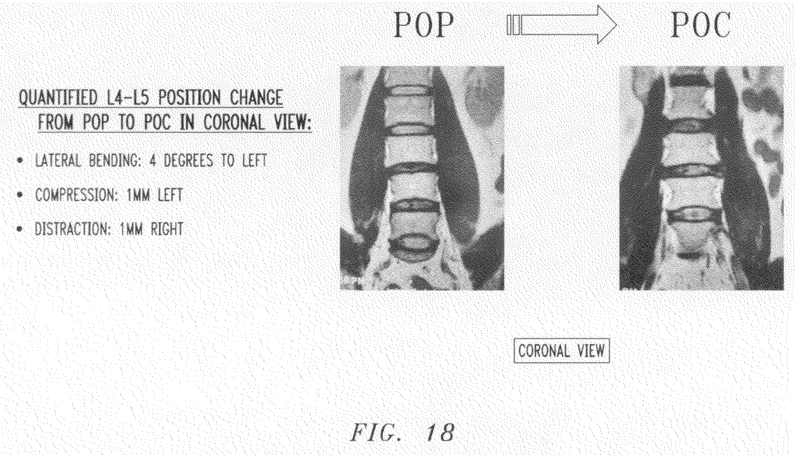

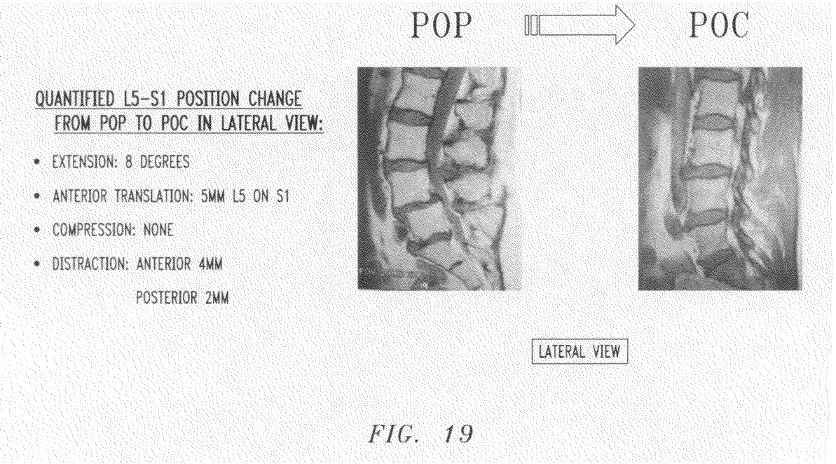

FIG. 17 illustrates the quantified changes in position (in degrees and millimeters) of L4-L5 from the POP to the POC in lateral view, and FIG. 18 shows the quantified changes in position of L4-L5 from the POP to the POC in coronal view. FIG. 19 illustrates the quantified changes in position of L5-S1 from the POP to the POC in lateral view.

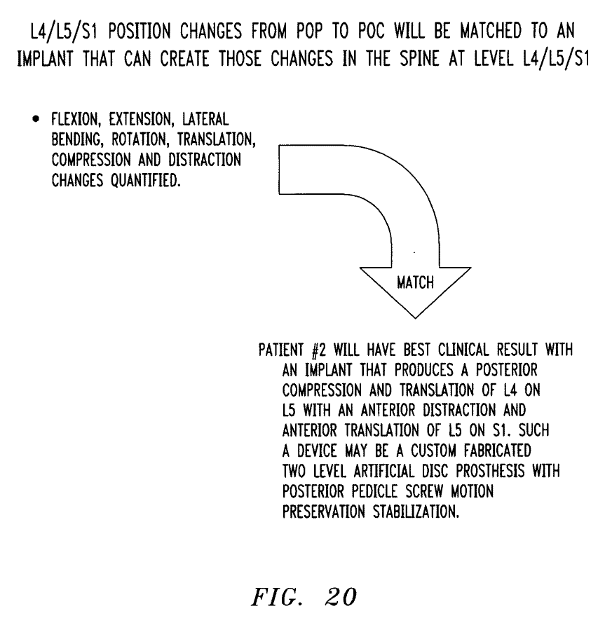

As shown in FIG. 20, the position changes in L4/L5/S1 from the POP to the POC for patient 2 are matched by the processing apparatus 106 to an implant that can create the changes in the spine at level L4/L5/S1. In this example, the output 110 of the apparatus 106 indicates that patient 2 should have the best clinical results with an implant that produces a posterior compression and translation of L4 on L5, with an anterior distraction and anterior translation of L5 on S1. The implant may be selected from among a number of implant devices whose characteristics are maintained in the system database 108, or the device may be a custom fabricated two level artificial disc prosthesis with posterior pedicle screw motion preservation stabilization.

The implant device(s) required to achieve the correct POC for any patient may also be custom fabricated by way of balloons that are placed at determined locations between the adjacent vertebra at each level to be treated, and then inflated to achieve the desired correction.

While the foregoing represents preferred embodiments of the invention, it will be understood by those skilled in the art that various modifications and changes may be made without departing from the spirit and scope of the invention, and that the invention includes all such modifications and changes as come within the scope of the appended claims.

I claim:

1. A system for determining spinal implants, comprising:
a surgical table constructed and arranged to move a patient when secured on the table into a number of different positions in response to corresponding commands by the patient, the positions including a position of pain (POP) where the patient experiences a maximum pain from one or more affected levels of his or her spine, and a position of comfort (POC) where the patient experiences a least amount of pain from the affected levels;
a scanner operatively associated with the table for scanning the patient at least at the POP and the POC, and for producing corresponding radiographic image data corresponding to the patient's spine; and
image processing apparatus coupled to the scanner and configured to quantify changes in relative position of adjacent vertebra at each affected level when the vertebra move from a configuration corresponding to the patient's POP to a configuration corresponding to the patient's POC, according to the image data produced by the scanner.

2. A system according to claim 1, including an implant device database associated with the image processing apparatus, wherein the database is configured to store information concerning characteristics of a number of implant devices selected from the group comprising artificial discs, rod/screw constructs, flexible rods, and distraction devices.

3. The system of claim 2, wherein the image processing apparatus is programmed and configured to select one or more implant devices from among the devices whose characteristics are stored in the database, according to the quantified changes in position of the vertebra at each affected level.
